(19) United States
(12) Patent Application Publication
(10) Pub. No.: US 2014/0066858 A1
(43) Pub. Date: Mar. 6, 2014

(54) VACUUM-OPERATED RETRACTABLE SAFETY SYRINGE

(71) Applicant: Strategic Product Development, Inc., Lincoln, MA (US)

(72) Inventors: Richard H. Theriault, Lincoln, MA (US); Andrew Goddard, Beverly, MA (US); Orlando Soto, Salem, MA (US); Sean J. Albert, Barrington, NH (US)

(73) Assignee: Strategic Product Development, Inc., Lincoln, MA (US)

(21) Appl. No.: 14/071,495

(22) Filed: Nov. 4, 2013

Related U.S. Application Data
(63) Continuation of application No. PCT/US12/36195, filed on May 2, 2012.
Application No. PCT/US12/36195 is a continuation of application No. PCT/US12/36178, filed on May 2, 2012.
(60) Provisional application No. 61/481,610, filed on May 2, 2011.
(60) Provisional application No. 61/587,654, filed on Jan. 17, 2012.
(60) Provisional application No. 61/481,610, filed on May 2, 2011.

(51) Int. Cl. A61M 5/32

(52) U.S. Cl. CPC A61M 5/3234
USPC 604/198; 29/428

(57) ABSTRACT

Vacuum-operated safety syringes designed to prevent an accidental needle prick, where the needle of the syringe is retracted after use into a vacuum created within the hollow plunger and/or the syringe barrel of the syringe. Retraction of the needle assembly is accomplished from minimal moving parts according to an exceedingly simple design. In some embodiments, one or more one-way valves prevent airflow into the interior of the syringe barrel and thereby maintain the vacuum that is created during the act of injection. In some embodiments, the plunger of the safety syringe is manufactured to be pre-conditioned at negative pressure.

### RELATED APPLICATIONS

This application is a Continuation of International Application No. PCT/US2012/036195, filed on May 2, 2012 and entitled VACUUM-OPERATED RETRACTABLE SAFETY SYRINGE, which claims the benefit of U.S. Provisional Application No. 61/481,610, filed on May 2, 2011 and entitled VACUUM-OPERATED RETRACTABLE SAFETY SYRINGE, and U.S. Provisional Application No. 61/587,654 filed on Jan. 17, 2012 and entitled VACUUM-OPERATED RETRACTABLE SAFETY SYRINGE; and is a Continuation of International Application No. PCT/US2012/036178, filed on May 2, 2012 and entitled VACUUM-OPERATED RETRACTABLE SAFETY SYRINGES, which also claims the benefit of U.S. Provisional Application No. 61/481,610, filed on May 2, 2011 and entitled VACUUM-OPERATED RETRACTABLE SAFETY SYRINGE. The entire teachings of the above applications are incorporated herein by reference.

### BACKGROUND

Hypodermic syringes are commonly used in the healthcare industry to inject therapeutics below the skin of patients. Despite their utility, hypodermic syringes present significant safety risks to healthcare practitioners, patients, and anyone who could accidentally be pricked by the exposed needle of a hypodermic syringe. The resulting injury could be as simple as a minor skin laceration, or as deadly as an infection from a virus in the patient's blood.

Numerous individuals are at risk of injury from injuries resulting from the exposed needle of a hypodermic syringe. Healthcare practitioners are exposed to this danger in routine medical practice. Diabetics and others who self-administer daily injections are at risk, as are members of their household. After disposal, conventional syringes may continue to pose a risk to sanitation workers and anyone else who comes in contact with landfills and waste management processes. Some syringes will undoubtedly be disposed of or handled improperly prior to disposal, increasing the chance of injury. Thus, conventional syringes can pose a danger to healthcare practitioners and others.

Several hypodermic syringes with retractable needles have been developed. The retraction of the needle into the barrel of the syringe after use reduces the risk of “needle prick”, or the accidental pricking of the person giving the injection after using the syringe.

Several types of syringes have been developed which accomplish the retraction in different ways. Examples include the needle syringes disclosed in U.S. Pat. No. 4,692,156 (Haller); U.S. Pat. No. 4,675,005 (DeLuccia); U.S. Pat. No. 4,747,830 (Gloyer, et al.); U.S. Pat. No. 4,790,822 (Haining); and U.S. Pat. No. 4,950,251 (Haining). These syringes disclosed include a hypodermic needle mounted on a carrier which slides in the barrel. A plunger is locked to this carrier after the injection has been given and the needle and carrier is withdrawn up into the barrel by withdrawal of the plunger.

Other automatic retractable needle syringes include loaded springs which, when released, retract the needle into the barrel. One example of this type is U.S. Pat. No. 5,885,257 (Badger). Further, vacuum powered retraction mechanisms are disclosed in U.S. Pat. No. 5,000,736 (Kaufhold et al.) and U.S. Pat. No. 6,846,301 (Smith et al.). Importantly, each of the vacuum powered retraction mechanisms in the patents of Kaufhold et al. and Smith et al. is relatively complex. In the syringes described by Kaufhold et al., the vacuum is provided in the syringe as shipped and breaking a seal activates the mechanism. In syringes described by Smith et al., the vacuum is created by movement of the plunger within the barrel.

Each reference, U.S. patent, and U.S. patent application that is disclosed herein is incorporated by reference in its entirety.

### SUMMARY

One disadvantage of prior art carrier-mounted needle syringes is that the intricate locking mechanism is relatively complex and takes up some space in the barrel of the syringe and may prevent all of the measured liquid from being ejected by the plunger, and/or limit the space available for retaining the retracted needle (thereby limiting needle size). This problem is exacerbated in the very small syringes such as the 1 cc tuberculin type. The liquid left in the barrel may be a substantial portion of the measured dose. In addition, the narrowness of the barrel of the 1 cc syringe makes it difficult to design a needle carrier and locking mechanism that will fit in the barrel without enlarging the diameter so much as to make the calibration useless.

One drawback to the invention by Smith et al. is that the entire volume of the barrel has to be evacuated and the resulting force that can be applied over the entire cross sectional area is relatively small. Further, the retraction mechanism of Smith et al. requires numerous moving parts that may contribute to increased manufacturing costs and limit the space in the needle barrel available to be used to retain the needle (thus limiting needle size). For example, the vacuum created by the syringes of Smith et al. is prone to failure. This is so because the vacuum is maintained by a stopper that primarily serves as a seal located between the exterior of the plunger and the interior of barrel. Apart from being a seal, this stopper of Smith et al. is further intended to function as a one-way valve to minimize the loss of vacuum created within the interior of barrel. However, leakage of vacuum can occur through the stopper-based one-way valves of Smith et al. Consequently, a vacuum-operated safety syringe is needed that benefits from a simpler and more cost-effective design. In particular, a vacuum-operated safety syringe is needed that utilizes superior one-way valves to more effectively maintain a vacuum within the syringe and thereby improve the ability of the syringe to safely and effectively retract the needle assembly.

To address the deficiencies in the prior art, a particular safety syringe includes a vacuum-operated retractable needle that benefits from a simple design that requires minimal moving parts. Also, particular one-way valves are superior to such valves currently used in existing syringes. The one-way valves that are incorporated into the present safety syringes more effectively maintain a vacuum within the syringe and thereby improve the ability of the syringe to safely and effectively retract the needle assembly. The safety syringe can be produced at low cost relative to existing syringes, and a large portion of the syringe can be available to retain the retracted needle, allowing for the use of larger needle sizes. Another significant advantage of the present safety syringe is that it can be safely and reliably operated relative to existing syringes by a healthcare practitioner. In some embodiments, the safety syringe can also be used in non-medical applications, such as chemical handling processes and or in analytical applications.

The vacuum-operated safety syringe of the present disclosure retracts its needle into the interior of a hollow plunger or syringe barrel to prevent the healthcare practitioner from accidentally getting stuck by the exposed needle. The retractable needle protects various people, including healthcare practitioners, their patients, and sanitation workers involved with disposal of medical waste. The safety syringe can prevent or reduce injuries ranging from minor skin lacerations to serious contamination by medications, germs, or viruses. In some embodiments, the syringe is a disposable, single-use device, and can be available in various sizes and shapes. It is an object of the present invention to provide an improved vacuum-operated safety syringe with a retractable-needle.

In one aspect, the safety syringe includes a retractable needle assembly, a syringe barrel for the withdrawal or injection of fluids, and plunger handle posts (e.g., of non-hollow shape) that fit into the syringe barrel. In some embodiments, negative pressure (i.e., vacuum) is created within the interior of the syringe barrel during the act of injection as the plunger handle posts are depressed through the syringe barrel towards the retractable needle assembly. In some embodiments, one or more one-way valves prevent airflow into the interior of the syringe barrel and thereby maintain the vacuum that is created during the act of injection, while no positive pressure is built up during loading of the syringe. Upon complete depression of the plunger handle posts through the syringe barrel, the retractable needle assembly is drawn into the syringe barrel by the vacuum created in the interior of the syringe barrel.

In one aspect, the safety syringe includes a retractable needle assembly, a syringe barrel for the withdrawal or injection of fluids, and a hollow plunger (e.g., of cylindrical shape) that fits into the syringe barrel. In some embodiments, negative pressure is created within the interior of the syringe barrel during the act of injection as the hollow plunger is depressed through the syringe barrel towards the retractable needle assembly. One or more one-way valves prevent airflow into the interior of the syringe barrel and thereby maintain the vacuum that is created during the act of injection (while avoiding positive pressure build up during loading of the syringe). Upon substantial or complete depression of the hollow plunger through the syringe barrel, one or more orifice(s) in the wall of the hollow plunger enter the interior of the syringe barrel. Thus, the negative pressure of the interior of the syringe barrel evacuates air from the interior of the cylindrical plunger and distributes negative pressure throughout both the syringe barrel and the cylindrical plunger. Upon engagement of the hollow syringe barrel with the retractable needle assembly, the retractable needle assembly is drawn into the interior of the hollow plunger by the vacuum created in the hollow plunger or syringe barrel by the act of injection.

In one aspect, the safety syringe includes a retractable needle assembly, a syringe barrel for the withdrawal or injection of fluids, and a plunger that fits into the syringe barrel, where the interior of the plunger is manufactured to be at negative pressure. Thus, in some embodiments, negative pressure (i.e., vacuum) is incorporated within the interior of the plunger at the time of manufacture rather than being created during the act of injection. Upon complete depression of the plunger through the syringe barrel, and engagement of the syringe barrel with the retractable needle assembly, the retractable needle assembly is drawn into the interior of the plunger by the pre-existing negative pressure that was incorporated within the interior of the plunger at the time of manufacture.

Healthcare practitioners can operate the presently disclosed safety syringe in the conventional manner that syringes are used to inject a patient. A plunger is used to draw fluid into the syringe and inject fluid from the syringe into the patient. During injection of the patient, the plunger and a one-way valve gradually generate a vacuum within the plunger or syringe barrel. Following injection, the vacuum within the plunger automatically pulls the piston toward the rear end of the plunger, retracting the needle safely into the interior of the plunger or syringe barrel.

### BRIEF DESCRIPTION OF THE DRAWINGS

The foregoing and other objects, features and advantages of the invention will be apparent from the following more particular description of particular embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-1C are schematics of a particular safety syringe that retracts its needle into the interior of the syringe barrel in accordance with a particular embodiment of the invention.

FIGS. 2A-2C are schematics of a particular safety syringe that retracts a cannula assembly into a hollow plunger in accordance with a particular embodiment of the invention.

FIGS. 3A-3C are schematics of a particular safety syringe having a one-way purge valve that retracts a cannula assembly into a hollow plunger in accordance with a particular embodiment of the invention.

FIGS. 4A-4C are schematics of another particular safety syringe having a one-way valve that retracts a cannula assembly into a hollow plunger in accordance with a particular embodiment of the invention.

FIGS. 5A-5C are schematics of yet another safety syringe that retracts a cannula assembly into a hollow plunger in accordance with a particular embodiment of the invention.

FIGS. 6A-6C are schematics of a safety syringe having a hollow plunger that is preconditioned at negative pressure for retracting a cannula assembly into the inner diameter of plunger in accordance with a particular embodiment of the invention.

FIGS. 7A-7C are schematics of a safety syringe that is pre-filled with liquid, and features an initially retraced, deployable needle in accordance with a particular embodiment of the invention.

### DETAILED DESCRIPTION

The present disclosure relates generally to vacuum-operated safety syringes designed to prevent an accidental needle prick, where the needle of the syringe is retracted after use into a vacuum created within the hollow plunger or the syringe barrel of the syringe. Particular syringes feature retraction of the needle assembly using minimal moving parts according to an elegant design. In various embodiments, negative pressure (i.e., vacuum) is created within a hollow plunger (e.g., of cylindrical shape) or the interior of the syringe barrel during the act of injection as the plunger is depressed through the syringe barrel towards the retractable needle assembly. Upon complete depression of the plunger through the syringe barrel, the retractable needle assembly is drawn into the syringe barrel by the vacuum created in the interior of the syringe barrel. In some embodiments, one or more one-way valves are utilized to prevent airflow into the interior of the syringe barrel and thereby maintain the vacuum that is created during the act of injection. These one way valves can also operate to allow air to escape from the syringe during loading, thereby reducing or elimination unwanted build up of positive pressure. In some embodiments, the plunger of the safety syringe is manufactured to be pre-conditioned at negative pressure.

For the purposes of the present disclosure, the term “plastic” refers to a class of materials including injection molded polypropylene plastic, synthetic resins, polyolefin resins such as polyethylene and polypropylene, polyvinyl chloride, PET (polyethylene terephthalate), EVA (ethylene-vinyl acetate copolymer), EVOH (ethylene-vinyl alcohol copolymer), polyamide, polyvinylidene chloride, polyvinyl fluoride, polytrifluorochloroethylene, polyester, nylon, mixtures thereof and laminated bodies thereof, the material is not limited especially if it is known for use as a medical equipment material and does not interact with the medicine or liquid accommodated in the barrel of the syringe.

The term “elastomer” refers to elastic materials having sealing property (in other words, gas-tight property) can be used. Thus the following elastomers can be used: injection molded thermoplastic elastomer, natural rubber, isoprene rubber, butyl rubber, butadiene rubber, styrene-butadiene rubber, silicone rubber, styrene-butadiene-styrene block copolymer, hydrogenated styrene-butadiene copolymer, hydrogenated styrene-ethylene-butylene-styrene block copolymer, ethylene-α-olefin copolymer rubber, thermoplastic elastomer, elastomers of polyurethane family, elastomers of polyamide family, elastomers of polyester family, vulcanized isoprene rubber, vulcanized butyl rubber, vulcanized styrene-butadiene-styrene block copolymer, and mixtures of these substances.

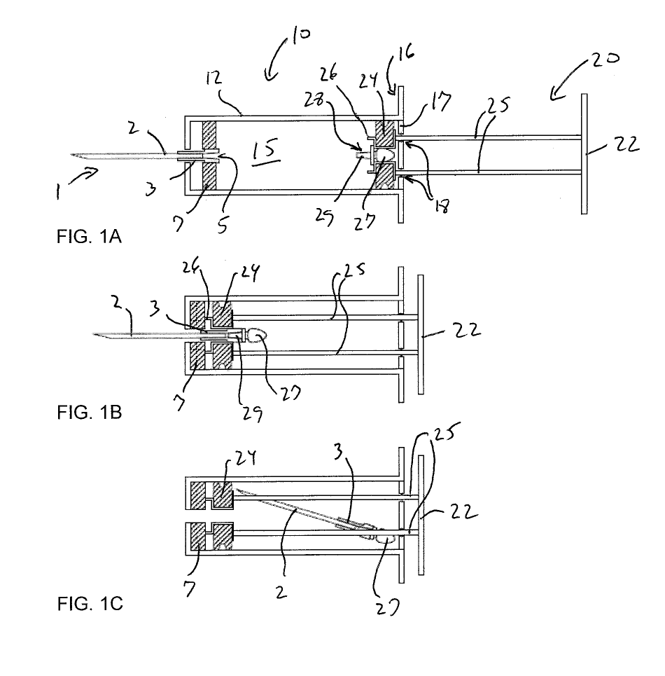

FIGS. 1A-1C are schematics of a particular safety syringe that retracts its needle into the interior of the syringe barrel in accordance with a particular embodiment of the invention. As shown, the safety syringe includes a retractable cannula or needle assembly 1 for receiving or delivering fluids, a syringe barrel assembly 10 for storing withdrawn or injection fluids, and a plunger assembly 20 for actuating the syringe.

The needle assembly 1 includes a needle 2 and cylindrical hub 3, which is terminated by a receiving port 5. The cannula 2 can be of various sizes. The hub 3 is held within the barrel 10 by a washer 7 at the distal end of the barrel assembly 10. In some embodiments, the cannula 2 is permanently attached to the hub 3. In some embodiments, the proximal end of the cannula assembly 1 is positioned in the center of a washer 7. The washer 7 creates a water tight seal at the hub 3 and syringe body 12.

The barrel assembly 10 includes a cylindrical body 12 surrounding an inner cavity 15. A cannula orifice 11 receives the needle hub 3 to allow the needle to form a passageway from the interior cavity 15. Also shown are a finger flange 16 and an end cap 17. The end cap includes end cap seals 18 that allow the plunger assembly 20 to move longitudinally.

The plunger assembly 20 includes a plunger handle posts 25 (e.g., of non-hollow shape) extending from a thumb rest 22. The plunger handle posts 25 fit into the syringe barrel 10 through the end cap seals 18. In some embodiments, the plunger handle posts 25 are cylindrical. In some embodiments, negative pressure (i.e., vacuum) is created within the interior cavity 15 of the syringe barrel 10 during the act of injection as the plunger handle posts 25 are depressed through the syringe barrel 10 towards the retractable needle assembly 1. In some embodiments, one or more one-way valves prevent airflow into the interior cavity 15 of the syringe barrel 10 and thereby maintain the vacuum that is created during the act of injection. Upon complete depression of the plunger handle posts 25 through the syringe barrel 10, the retractable needle assembly 1 is drawn into the internal cavity 15 of the syringe barrel 10 by the vacuum created in the interior cavity 15 of the syringe barrel 10 by the act of injection.

In some embodiments, a needle catch assembly 28 includes a needle catch 29, which can be permanently attached to a piston bung 27, held within a needle catch adapter 26. In some embodiments, a barrel seal 24 is affixed to the needle catch adapter 26. The needle catch assembly 28 is held in the inner diameter of the needle catch adapter 26. The bung 27 creates an air tight seal with the inner diameter of the needle catch adapter 26. The size of features on needle catch 29 is such that there is some amount of interference with the inner diameter of the needle catch adapter 26. In some embodiments, the syringe barrel end cap 17 is affixed to proximal end of the syringe barrel 10 to provide an airtight seal between the two. In some embodiments, the plunger handle posts 25 can be affixed to needle catch adapter 26. In some embodiments, the plunger handle posts 25 can be supplied loose from the needle catch adapter 26. If desired, the plunger handle posts 25 are affixed to needle catch adapter 26 by inserting the posts 25 through end cap seals 18. The end cap seals 18 create air tight seals with the plunger handle posts 25.

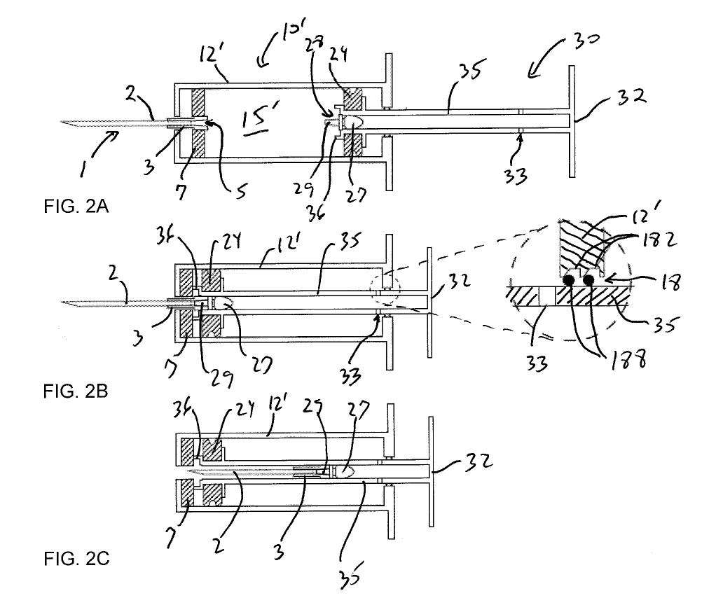

As illustrated, the safety syringe of FIGS. 1A-1C is designed to retract the cannula 2 into the interior cavity 15 of the syringe barrel 10 upon complete depression of plunger handle posts 25 by the user. The retraction of the cannula 2 is accomplished by the creation of a vacuum in the interior 15 of the syringe barrel 10 upon depression of plunger handle posts 25 by the user. In some embodiments, one or more one-way valving is used to create a vacuum in the interior 15 of the syringe barrel 10 during depression of the plunger 20 while allowing air to escape during retraction of the plunger 20 (e.g., during loading of the syringe from a liquid resevoir, not shown). For example, the end cap 17 can have one-way valving to allow for purging of trapped air in the interior 15 of the syringe barrel 10. The one way valving enables air to exit the syringe barrel 10 upon retraction of the plunger handle posts 25 away from the cannula 2, but air cannot enter the syringe barrel 10 upon depression of the plunger handle posts 25 toward the cannula 2. Thus, depression of the plunger handle posts 25 creates a vacuum in the interior of the syringe barrel 10 upon complete depression of plunger handle posts 25 by the user, which thereby draws the cannula 2 into the interior cavity 15 of syringe barrel 10. In some embodiments, the airtight seals 18 can be one way valve type seals (e.g., of the type illustrated in FIG. 2B), to provide the one way valving.

For example, upon filling the syringe barrel cavity 15 with the liquid to be injected, the plunger handle posts 25 are retracted away from the cannula 2. Upon subsequent injection of liquid, the plunger handle posts 25 are then depressed toward the cannula 2, where the needle catch 29 engages the receiving port 5 of the hub 3 and becomes affixed, as shown in FIG. 1B. As the plunger 20 is depressed negative pressure is created on the proximal side of the barrel seal 24. While depressing the plunger 20, a shoulder feature on the needle catch adapter 26 will displace the washer 7 so that the cannula assembly 1 is free to move.

Immediately after release of the cannula assembly 1 from the washer 7, further depression of the plunger handle posts 25 in the distal direction will push the needle catch 29 through the inner diameter of needle catch adapter 26 thus releasing the needle catch bung 27 along with the affixed cannula assembly 1. At this time the needle catch bung 27 and cannula assembly 1 will be aspirated into the interior cavity 15 of the syringe barrel 10 portion, which is proximal to the barrel seal 24 by the negative pressure previously created, as shown in FIG. 1C. Note that, aside from a small volume occupied by the plunger handle posts 25, nearly the entire interior volume of the syringe barrel 10 is available for retention of the cannula assembly 1.

The safety syringe of FIGS. 1A-1C, as well as those that follow, can be assembled from components made from a variety of materials. In some embodiments, the cannula 2 is made of stainless steel tubing (e.g., medical hypodermic stainless steel tubing). In some embodiments, the cannula 2 is of various sizes (e.g., in the range of fractions of a centimeter or less to tens of centimeters or more). In some embodiments, the hub 3 is made of plastic (e.g., injection molded polypropylene plastic). In some embodiments, the cannula 2 can be assembled to the hub 3 by any method known to one of ordinary skill (e.g., over molding, UV glue bonding, or other any other bonding method). In some embodiments, washer 7 is made of an elastomer (e.g., injection molded thermoplastic elastomer). In some embodiments, the syringe barrel body 12 is made of plastic (e.g., polypropylene plastic or injection molded polypropylene plastic). In some embodiments, the needle catch 29 is made of plastic (e.g., polypropylene plastic or injection molded polypropylene plastic). In some embodiments, the bung 27 is made of an elastomer (e.g., injection molded thermoplastic elastomer). In some embodiments, the bung 27 can be molded separately. In some embodiments, the bung 27 can be overmolded onto needle catch 29. In some embodiments, the needle catch adapter 26 is made of plastic (e.g., polypropylene plastic or injection molded polypropylene plastic). In some embodiments, the barrel seal 24 is made of an elastomer (e.g., injection molded thermoplastic elastomer). In some embodiments, the barrel seal 24 can be molded separately. In some embodiments, the barrel seal 24 can be overmolded onto needle catch adapter 26. In some embodiments, syringe barrel end cap 17 is made of plastic (e.g., polypropylene plastic or injection molded polypropylene plastic). In some embodiments, end cap seal 18 is made of an elastomer (e.g., injection molded thermoplastic elastomer). In some embodiments, end cap seal 18 is made of rubber. In some embodiments, the plunger posts 22 and thumb rest 22 are made of plastic (e.g., polypropylene plastic or injection molded polypropylene plastic).

Referring to FIGS. 2-5, various embodiments of safety syringes include a retractable needle assembly, a syringe barrel for the withdrawal or injection of fluids, and a hollow plunger (e.g., of cylindrical shape) that fits into the syringe barrel. In some embodiments, negative pressure is created within the interior of the syringe barrel during the act of injection as the hollow plunger is depressed through the syringe barrel towards the retractable needle assembly. One or more one-way valves prevent airflow into the interior of the syringe barrel and thereby maintain the vacuum that is created during the act of injection, while allowing air to escape during the loading of the syringe. Upon substantial or complete depression of the hollow plunger through the syringe barrel, one or more orifice(s) in the wall of the hollow plunger enter the interior of the syringe barrel. Thus, the negative pressure of the interior of the syringe barrel evacuates air from the interior of the cylindrical plunger and distributes negative pressure throughout both the syringe barrel and the cylindrical plunger. Upon engagement of the hollow syringe barrel with the retractable needle assembly, the retractable needle assembly is drawn into the interior of the hollow plunger by the vacuum created in the hollow plunger or syringe barrel by the act of injection.

FIGS. 2A-2C are schematics of a particular safety syringe that retracts a cannula assembly into a hollow plunger in accordance with a particular embodiment of the invention. The particular safety syringe includes a cannula 2, which is permanently attached to a hub 3 to form a cannula assembly 1. The cannula assembly 1 is located at the distal end of a syringe barrel 10′ having a generally cylindrical-shaped body 12′ defining an inner cavity 15′. The proximal end of the cannula assembly 1 is positioned in the center of a washer 11. The washer 11 creates a water tight seal at the hub 3 and syringe barrel 10′.

As described above, a needle catch 29 is permanently attached to piston bung 27 to form a needle catch assembly 28. A barrel seal 24 is affixed to a plunger 20 having a plunger shaft 35 and a thumb rest 32. The needle catch assembly 28 is held in the inner diameter of a plunger shaft 35. The bung 27 creates an air tight seal with he inner diameter of the plunger shaft 35. The size of features on the needle catch 29 is such that there is some amount of interference with the distal orifice diameter of the plunger shaft 35.

The proximal end of the syringe barrel 10′ where the plunger shaft 35 passes through can be a separate piece assembled to the majority of the syringe barrel 10′ to provide an airtight seal between the two pieces. The plunger shaft 35 passes through one, two, or more shaft seals 18 to provide an air-tight seal between the syringe barrel body 12′ and the plunger shaft 35.

As shown in FIG. 2B, the shaft seal 18 includes o-rings 188 retained in respective grooves 182 in the syringe body 12′ to provide an air tight seal when negative pressure is created in the internal volume of syringe barrel 10 by the plunger 30 moving in the distal direction. The geometry of the groove 182 and the o-ring shaft seal 188 is such that the increase of negative pressure created when the plunger 30 is moved in the distal direction causes the airtight seal to become more robust. The geometry of the groove 182 and the o-ring shaft seal 188 is such that trapped air can be released when the plunger is actuated in a proximal direction.

In some embodiments, the safety syringe is designed to retract the cannula assembly 1 into the internal volume of the plunger shaft 35 upon complete depression of the plunger 30 by the user.

As shown in FIG. 2B, after filling of the device with liquid, the plunger 30 is depressed where the needle catch 29 engages the hub 3 and becomes affixed. As the plunger 30 is depressed negative pressure is created on the proximal side of the barrel seal 18. Prior to release of the cannula assembly 1, an orifice 33 into the plunger shaft 35 enters the negative pressure volume of the syringe barrel 10 joining the negative pressure volume of the syringe barrel 10 and the internal volume of plunger shaft 35.

As shown in FIG. 2C, while depressing the plunger 30 the shoulder feature 36 on the plunger shaft 35 will displace the washer 7 so that the cannula assembly 1 is free to move. Immediately after release of the cannula assembly 1 from the washer 7, further travel of the plunger handle in the distal direction will push the needle catch 29 through the distal orifice diameter of the plunger shaft 35 thus releasing the needle catch assembly 28 along with the affixed cannula assembly 1. At this time the needle catch assembly 28 and cannula assembly 1 will be aspirated into the plunger shaft 35 portion which is proximal to the distal orifice in the plunger 30 by the negative pressure previously created. Note that nearly the entire interior volume of the plunger shaft 35 is available for retaining the cannula assembly 1, allowing for the use of large cannula sizes.

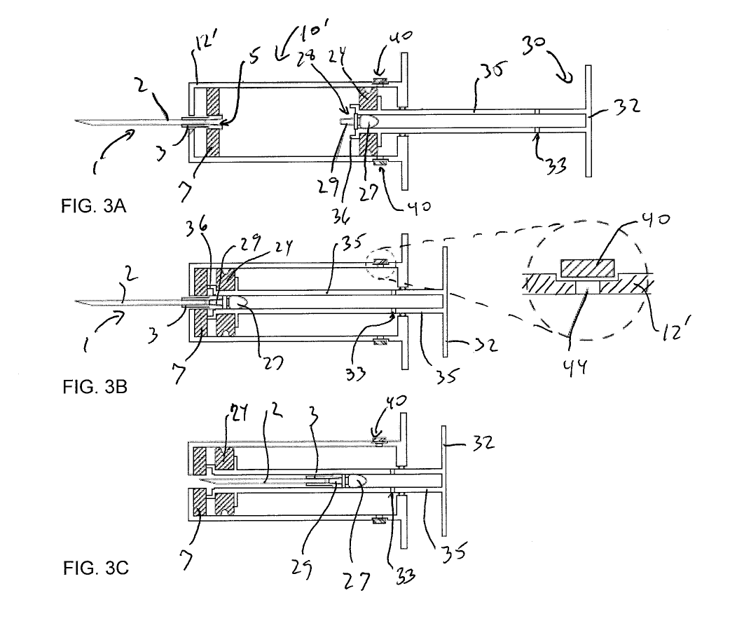

FIGS. 3A-3C are schematics of a particular safety syringe having a one-way purge valve that retracts a cannula assembly into a hollow plunger in accordance with a particular embodiment of the invention. The cannula assembly 1 and plunger assembly 30 are substantially identical to those in FIGS. 2A-2C. Furthermore, the syringe barrel 10′ is substantially similar to the syringe barrel 10′ of FIGS. 2A-2C except for the inclusion of purge valves 40 at the proximal end.

The purge valve 40 is affixed to the syringe barrel 10′ covering orifice 44 through the syringe barrel body 12′ as best shown in FIG. 3B. The purge valve 40 provides an airtight seal when negative pressure is created in the internal volume of the syringe barrel 10′ that is proximal to the barrel seal 24, but the seal is released when positive pressure is created in that volume. In some embodiments, the purge valve 10 is made of an elastomer (e.g., injection molded thermoplastic elastomer). In some embodiments, the purge valve 10 is made of plastic (e.g., injection molded plastic, polypropylene plastic, or injection molded polypropylene plastic). In some embodiments, the purge valve 10 is made of metal (e.g., steel or spring type steel) with an elastomeric material laminated to the inside. In some embodiments, the purge valve 10 is made of metal (e.g., steel or spring type steel) and overmolded with an elastomer (e.g., injection molded thermoplastic elastomer).

As shown, the safety syringe is designed to retract the cannula assembly 1 into the internal volume of the plunger shaft 35 upon complete depression of plunger 30 by the user. The process is generally described above with reference to FIGS. 2A-2C. However, prior to release of the cannula assembly 1, the purge valve orifice 44 enters the negative pressure volume of the syringe barrel 10′ joining the negative pressure volume of the syringe barrel 10′ and the internal volume of the plunger shaft 35. Again, the needle catch assembly 28 and cannula assembly 1 will be aspirated in to the plunger shaft 35 portion which is proximal to the distal orifice in plunger shaft 35 by the negative pressure previously created. Again, nearly the entire interior volume of the plunder shaft 35 is available for retention of the cannula assembly 1.

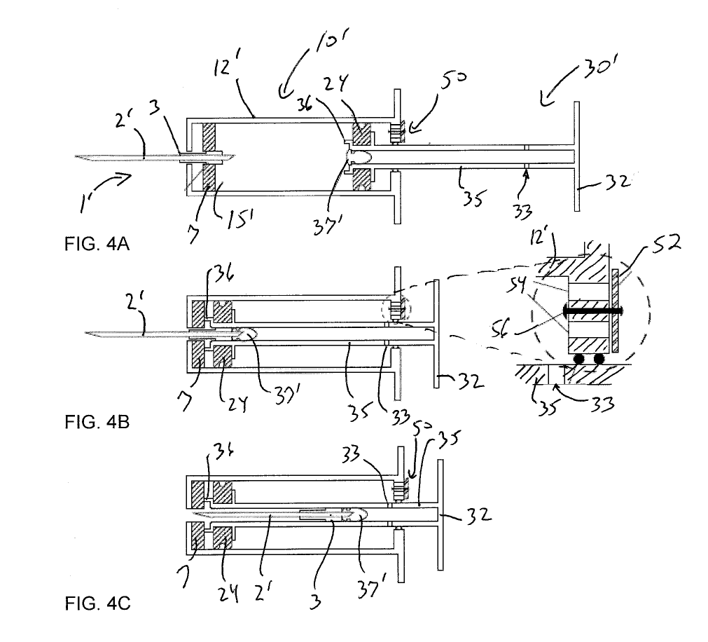

FIGS. 4A-4C are schematics of another particular safety syringe having a one-way valve that retracts a cannula assembly into a hollow plunger in accordance with a particular embodiment of the invention. In this embodiment, the catch mechanisms and the one-way valving differ from that of FIGS. 3A-3C.

As shown, the safety syringe 10′ includes a cannula assembly 1′ that includes a cannula 2′ permanently attached to a hub 3. The cannula assembly is located at the distal end of the syringe barrel 10′. The proximal end of the cannula assembly 1′ is positioned in the center of the washer 7, which creates a water tight seal at the hub 3 and syringe barrel body 12′. Unlike the previous embodiments, the cannula 2′ extends through the hub 3 and projects into the barrel cavity 15′.

To accommodate the cannula configuration, the plunger assembly 30′ is modified from that of FIGS. 3A-3B to include a different needle catch bung 37′. A barrel seal 24 is gain affixed to a plunger shaft 35. The needle catch bung 37′ is held at an inner diameter of the plunger shaft 35. The needle catch bung 37′ creates an air tight seal with the inner diameter of the plunger shaft 35.

The syringe barrel 10′ is substantially similar to that of FIGS. 3A-3C, except that it employs a different purge valve assembly 50. As shown, the purge valve assembly 50 is affixed to the proximal end of the syringe barrel 10′. The purge valve assembly 50 includes a purge valve 52 that can be attached to the syringe barrel 10′ with a valve retainer 56 or directly to the syringe barrel 10′. The purge valve 52 covers at least one orifice 54 through the syringe barrel body 12′ providing an airtight seal when negative pressure is created in the internal volume of the syringe barrel 10′ that is proximal to the barrel seal 24, but the seal is released when positive pressure is created in that volume.

In some embodiments, the safety syringe is designed to retract the cannula assembly 1′ into the internal volume of the plunger shaft 35 upon complete depression of the plunger 30′ by the user. After filling of the device, the plunger 30′ is depressed and the needle catch bung 37′ engages the cannula 2′ and becomes affixed. The needle catch bung 37′ and cannula 2′ can become affixed via piecing or other interlocking geometry. As shown, the cannula 2′ includes a sharp proximal end that pierces into (but not through) the bung 37′. In some embodiments, the bung 37′ can frictionally (or otherwise) engage the inner surface of the plunger shaft 35 to mechanically secure and retain the cannula assembly 1′ within the plunger shaft 35.

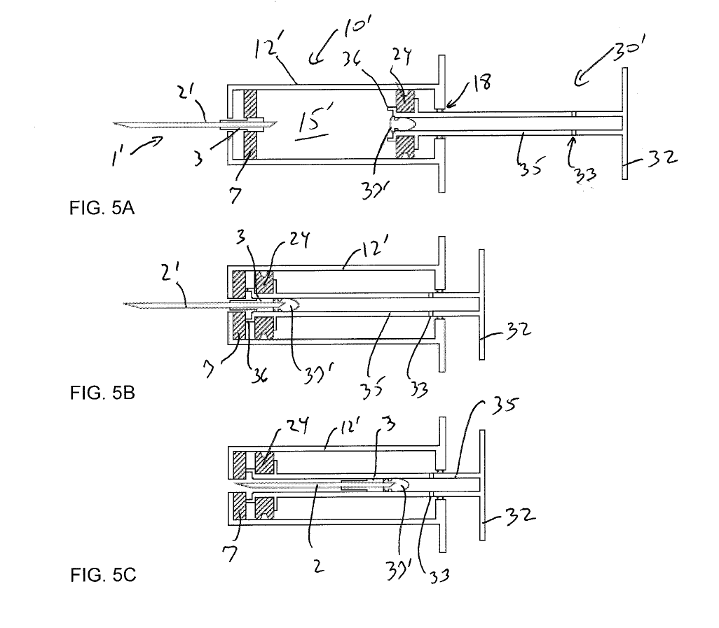

FIGS. 5A-5C are schematics of yet another safety syringe that retracts a cannula assembly into a hollow plunger in accordance with a particular embodiment of the invention. This embodiment operates similar to that of the safety syringe of FIGS. 2A-2C. The two embodiments differ in that the safety syringe of FIGS. 5A-5C employs the cannula assembly 1′ and plunger assembly 30′ of FIGS. 4A-4C.

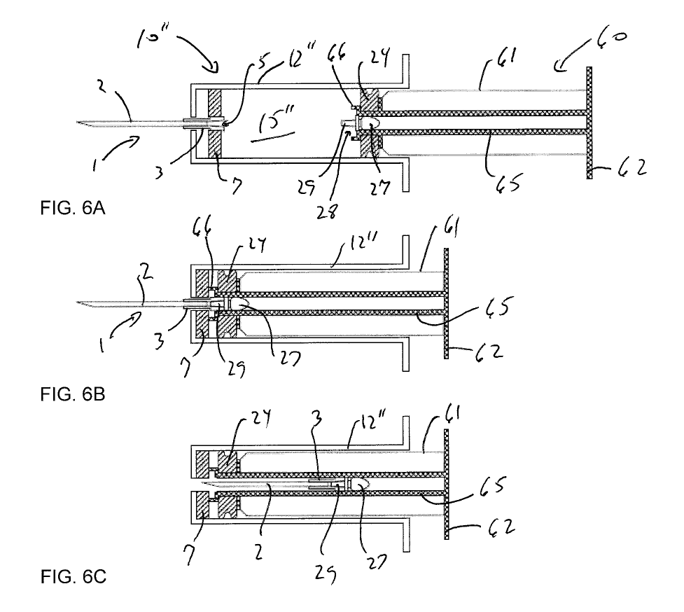

FIGS. 6A-6C are schematics of a safety syringe having a hollow plunger that is preconditioned at negative pressure for retracting a cannula assembly into the inner diameter of plunger in accordance with a particular embodiment of the invention.

As shown, in one embodiment, a safety syringe includes a retractable needle assembly 1 held by a washer 7, as previous described, a syringe barrel 10″ for the withdrawal or injection of fluids, and a plunger assembly 60 that fits into the syringe barrel 10″. In particular the plunger assembly 60 includes an outer cylindrical body 61 that is coaxial with an inner plunger shaft 65 terminated at the proximal end by a thumb rest 62. The plunger shaft 65 is terminated at the distal end by a cannula catch assembly 28, held within a plunger seal 24.

The interior of the plunger shaft 65 is manufactured at negative pressure. Thus, vacuum is preconditioned in the interior of the plunger prior to use. Thus, in some embodiments, negative pressure (i.e., vacuum) is incorporated within the interior of the plunger at the time of manufacture rather than being created during the act of injection. Upon complete depression of the plunger through the syringe barrel, and engagement of the syringe barrel with the retractable needle assembly, the retractable needle assembly is drawn into the interior of the plunger by the negative pressure that was incorporated within the interior of the plunger at the time of manufacture.

More particularly, the safety syringe includes a cannula assembly 1 that includes a cannula 2 permanently attached to a hub 2. The cannula assembly 1 are located at the distal end of the syringe barrel 10″. The proximal end of cannula assembly 1 is positioned in the center of a washer 7. The washer 7 creates a water tight seal at the hub 3 and the syringe barrel wall 10″.

At the distal end of the plunger assembly 60, a needle catch 29 is permanently attached to a piston bung 27. The barrel seal 24 is affixed to the plunger assembly 60. The needle catch assembly 28 is held in an inner diameter of the plunger shaft 65. The bung 27 creates an air tight seal an inner diameter of the plunger shaft 65. The size of features on the needle catch 29 is such that there is some amount of interference with the distal orifice diameter of the plunger shaft 65. Again, the internal volume of the plunger shaft 65 sealed with the piston bung 27 is at negative pressure.

In some embodiments, the safety syringe of FIG. 6 is designed to retract cannula assembly 1 into the internal volume of the plunger shaft 65 upon complete depression of the plunger 60 by the user. After filling of the device, the plunger 60 is depressed so that the needle catch 29 engages the hub 3 and becomes affixed, as shown in FIG. 6B. While depressing the plunger 60 the shoulder feature 66 on the plunger shaft 65 will displace the washer 7 so that the cannula assembly 1 is free to move.

Immediately after release of the cannula assembly 1 from the washer 7, further travel of the plunger handle in the distal direction will push the needle catch 29 through the distal orifice diameter of the plunger shaft 65 thus releasing the needle catch assembly 28 along with the affixed cannula assembly 1. At this time, as shown in FIG. 6C, the needle catch assembly 28 and cannula assembly 1 will be aspirated into the interior cavity of the plunger shaft 65 portion that is proximal to the distal orifice in the plunger shaft 65 by the supplied negative pressure.

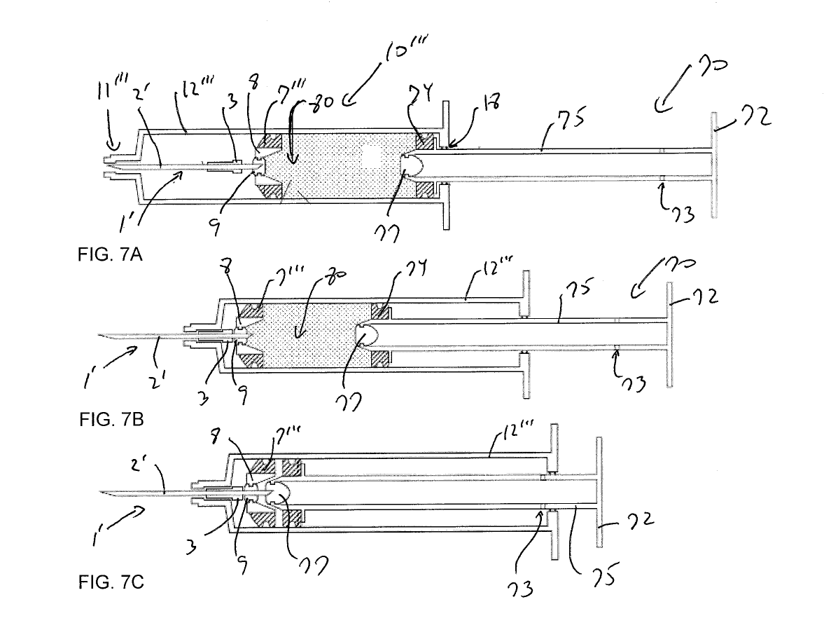

FIGS. 7A-7C are schematics of a safety syringe that is pre-filled with liquid, and features an initially retraced, deployable needle in accordance with a particular embodiment of the invention. As shown, in one embodiment a pre-filled safety syringes includes a retractable needle assembly 1′, a syringe barrel 10′″ for the withdrawal or injection of fluids, and a hollow plunger assembly 70 (e.g., of cylindrical shape) that fits into the syringe barrel 10′″. A retractable needle assembly 1′ can be pre-retraced, such that the device ships with the needle safely housed within the syringe body. In that case, the retractable needle assembly 1′ is initially deployed by depressing the plunger 70.

In some embodiments, negative pressure is created within the interior of the syringe barrel during the act of injection as the hollow plunger 70 is depressed through the syringe barrel towards the retractable needle assembly. One or more one-way valves prevent airflow into the interior of the syringe barrel 10′″ and thereby maintain the vacuum that is created during the act of injection. Upon substantial and/or complete depression of the hollow plunger 70 through the syringe barrel 10′″, one or more orifice(s) 73 in the wall of the hollow plunger shaft 75 enter the interior of the syringe barrel 10′″. Thus, the negative pressure of the interior of the syringe barrel evacuates air from the interior of the cylindrical plunger shaft 75 and distributes negative pressure throughout both the syringe barrel 10′″ and the cylindrical plunger 75. Upon engagement of the hollow syringe barrel with the retractable needle assembly, the retractable needle assembly is drawn into the interior of the hollow plunger shaft 75 by the vacuum created in the hollow plunger and/or syringe barrel by the act of injection.

For example, in one embodiment, a safety syringe includes a cannula 2. The cannula 2 is permanently attached to a hub 3 to form a cannula assembly 1′. The cannula assembly 1′ is initially located internal to the syringe barrel 10′″ at the distal end of the syringe barrel 10′″. The proximal end of the cannula 2 is held in the center of a needle shuttle bung 9. A syringe barrel feature 11′″ locates the needle assembly 1′ centrally to the syringe barrel 10′″. A needle shuttle seal 7′″ is permanently attached to a needle shuttle 8 to form a needle shuttle assembly. The needle shuttle bung 9 is held by the needle shuttle assembly creating a seal between the needle shuttle bung 9 and the syringe barrel wall 12′″.

A barrel seal 24 is affixed to the plunger shaft 75. A needle catch bung 77 is held in a certain size inner diameter of the plunger shaft 75. The needle catch bung 77 creates an air tight seal with a certain size inner diameter of the plunger shaft 75. The prefilled fluid 80 exists in the space between the needle shuttle assembly and the plunger assembly. The proximal end of the syringe barrel 10′″, which the plunger shaft 75 passes through can be a separate piece assembled to the majority of the syringe barrel 10′″ providing an airtight seal between the two pieces. The plunger shaft 75 passes through one, two or more shaft seals 18 to provide an air tight seal between the syringe barrel wall 12′″ and the plunger shaft 75. A purge valve as described above can be affixed to the proximal end of the syringe barrel 10′″.

Depressing the plunger 70 in a distal direction causes the fluid, shuttle assembly and cannula assembly to move in a distal direction until the hub 3 becomes engaged with the distal end of the needle shuttle bung 9. Depressing the plunger 70 further causes the proximal end of the cannula 1′ to pass through the needle shuttle bung 9, thus allowing the pre-filed fluid to pass through the internal diameter of cannula 1′. The needle shuttle bung 9 will come to rest after the cannula 1 has passed through it and may or may not be in contact with the hub 3 at this time.

As shown in FIG. 7C, the plunger 70 is depressed where the needle catch bung 77 engages the cannula 1′ and becomes affixed. The needle catch bung 77 and the cannula 1′ can become affixed via piecing or other interlocking geometry. At this time fluid flow through the inner diameter of cannula 1′ will stop. As the plunger 70 is depressed negative pressure is created on the proximal side of the barrel seal 24. Prior to release of the cannula assembly 1′, the orifice 73 on the plunger shaft 75 enters the negative pressure volume of the syringe barrel 10′″ joining the negative pressure volume of the syringe barrel 10′″ and the internal volume of the plunger shaft 75.

While depressing the plunger 70 further, features on plunger 70 or needle catch bung 77 will displace the shuttle assembly causing needle shuttle bung 9 to be released from the shuttle 8 so that the cannula assembly 1′ and attached needle shuttle bung 9 are free to move. After release of the cannula assembly 1 and the needle shuttle bung 9 further travel of the plunger 70 in the distal direction will push the needle catch bung 77 through the distal orifice diameter of plunger shaft 75 thus releasing the needle catch bung 77 along with the affixed cannula assembly 1′ and needle shuttle bung 9. At this time the needle catch bung 77, cannula assembly 1′ and needle shuttle bung 9 will be aspirated in to the plunger shaft 75 portion, which is proximal to the distal orifice in plunger 70 by the negative pressure previously created.

It should be understood that syringes of the types described herein can be used for any suitable purpose including, but not limited to, injections and or phlebotomy. Further details of the various embodiments are described in the incorporated U.S. Provisional Patent Applications.

While this invention has been particularly shown and described with references to particular embodiments, it will be understood by those skilled in the art that various changes in form and details may be made to the embodiments without departing from the scope of the invention encompassed by the appended claims. For example, various features of the embodiments described and shown can be omitted or combined with each other.

We claim:

1. A syringe comprising:
a retractable needle assembly;
a syringe barrel for the withdrawal or injection of fluids; and
a plunger handle post fittable into the syringe barrel, wherein negative pressure is created within the interior of the syringe barrel during an act of injection as the plunger handle post is depressed through the syringe barrel towards the retractable needle assembly.

2. The syringe of claim 1, further comprising a one-way valve that prevents airflow into an interior volume of the syringe barrel and thereby maintains negative pressure created during the act of injection, while no positive pressure is built up during loading of the syringe.

3. The syringe of claim 1, wherein upon complete depression of the plunger handle post through the syringe barrel, the retractable needle assembly is drawn into the syringe barrel by the negative pressure created in the interior of the syringe barrel.

4. The syringe of claim 1, wherein the retractable needle assembly is pre-retracted.

5. The syringe of claim 1, wherein the syringe barrel is pre-filled with a fluid.

6. A syringe comprising:
a retractable needle assembly;
a syringe barrel for the withdrawal or injection of fluids; and
a hollow plunger that fits into the syringe barrel;
wherein negative pressure is created within the interior of the syringe barrel during the act of injection as the hollow plunger is depressed through the syringe barrel towards the retractable needle assembly.

7. The syringe of claim 6, further comprising a one-way valve that prevents airflow into the interior of the syringe barrel and thereby maintains negative pressure created during the act of injection.

8. The syringe of claim 7, wherein upon substantial depression of the hollow plunger through the syringe barrel, an orifice in the wall of the hollow plunger enter the interior of the syringe barrel such that the negative pressure of the interior of the syringe barrel evacuates air from the interior of the cylindrical plunger and distributes negative pressure throughout both the syringe barrel and the cylindrical plunger to facilitate retraction of the needle assembly.

9. The syringe of claim 6, wherein the retractable needle assembly is pre-retract.

10. The syringe of claim 6, wherein the syringe barrel is pre-filled with a fluid.

11. A syringe comprising:
a retractable needle assembly;
a syringe barrel for the withdrawal or injection of fluids; and
a plunger that fits into the syringe barrel, where the interior of the plunger is manufactured to be at negative pressure.

12. The syringe of claim 11, wherein, upon substantial depression of the plunger through the syringe barrel, and engagement of the syringe barrel with the retractable needle assembly, the retractable needle assembly is drawn into the interior of the plunger by the pre-existing negative pressure that was incorporated within the interior of the plunger at the time of manufacture.

13. The syringe of claim 11, wherein the retractable needle assembly is pre-retracted.

14. The syringe of claim 11, wherein the syringe barrel is pre-filled with a fluid.

15. A method of making a syringe comprising:
assembling a retractable needle assembly;
forming a syringe barrel for the withdrawal or injection of fluids; and
fitting a plunger handle post into the syringe barrel, so that negative pressure is created within the interior of the syringe barrel during an act of injection as the plunger handle post is depressed through the syringe barrel towards the retractable needle assembly.

16. The method of claim 15, further forming a one-way valve that prevents airflow into an interior volume of the syringe barrel and thereby maintains negative pressure created during the act of injection, while no positive pressure is built up during loading of the syringe.

17. The method of claim 15, further comprising pre-retracting the retractable needle assembly.

18. The method of claim 15, further comprising pre-filling the syringe barrel with a fluid.

19. A method of making a syringe comprising:
assembling a retractable needle assembly;
forming a syringe barrel for the withdrawal or injection of fluids; and
fitting a hollow plunger into the syringe barrel;
wherein negative pressure is created within the interior of the syringe barrel during the act of injection as the hollow plunger is depressed through the syringe barrel towards the retractable needle assembly.

20. The method of claim 19, further comprising forming a one-way valve that prevents airflow into the interior of the syringe barrel and thereby maintains negative pressure created during the act of injection.

21. The method of claim 19, further comprising pre-retracting the retractable needle.

22. The method of claim 19, further comprising pre-filling the syringe barrel with a fluid.

23. A method of making a syringe comprising:
assembling a retractable needle assembly;
forming a syringe barrel for the withdrawal or injection of fluids; and
fitting a plunger into the syringe barrel, where the interior of the plunger is manufactured to be at negative pressure.

24. The method of claim 23, further comprising pre-retracting the retractable needle assembly.

25. The method of claim 23, further comprising pre-filling the syringe barrel with a fluid.
